(19) United States
(12) Patent Application Publication
(10) Pub. No.: US 2013/0090649 A1
(43) Pub. Date: Apr. 11, 2013

(54) DEVICE AND METHODS FOR RENAL NERVE MODULATION

(71) Applicant: Boston Scientific Scimed, Inc., Maple Grove, MN (US)

(72) Inventors: SCOTT R. SMITH, CHASKA, MN (US); MARK L. JENSON, GREENFIELD, MN (US); PATRICK A. HAVERKOST, BROOKLYN CENTER, MN (US)

(73) Assignee: BOSTON SCIENTIFIC SCIMED, INC., MAPLE GROVE, MN (US)

(21) Appl. No.: 13/647,129

(22) Filed: Oct. 8, 2012

Related U.S. Application Data
(60) Provisional application No. 61/545,937, filed on Oct. 11, 2011.

(51) Int. Cl. A61B 18/14

(52) U.S. Cl. 606/41

(57) ABSTRACT

Systems for nerve modulation are disclosed. An example system may include a first elongate element having a distal end and a proximal end and having at least one inflatable balloon and one nerve modulation element disposed adjacent the distal end. Expansion of the inflatable balloon may partially occlude a vessel and positions the nerve modulation element within the vessel.

### CROSS-REFERENCE TO RELATED APPLICATIONS

This application claims priority under 35 U.S.C. §119 to U.S. Provisional Application Ser. No. 61/545,937, filed Oct. 11, 2011, the entirety of which is incorporated herein by reference.

### TECHNICAL FIELD

The present invention relates to methods and apparatuses for nerve modulation techniques such as ablation of nerve tissue or other destructive modulation technique through the walls of blood tissue.

### BACKGROUND

Certain treatments require the temporary or permanent interruption or modification of select nerve function. One example treatment is renal nerve ablation which is sometimes used to treat conditions related to congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels and thus can be accessed intravascularly through the walls of the blood vessels. In some instances, it may be desirable to ablate perivascular renal nerves using a radio frequency (RF) electrode. However, such a treatment may result in thermal injury to the vessel wall at the electrode and other undesirable side effects such as, but not limited to, blood damage, clotting and/or protein fouling of the electrode. Increased cooling in the region of the nerve ablation may reduce such undesirable side effects. It is therefore desirable to provide for alternative systems and methods for intravascular nerve modulation.

### SUMMARY

The disclosure is directed to several alternative designs, materials and methods of manufacturing medical device structures and assemblies for partially occluding a vessel and performing nerve ablation.

Accordingly, one illustrative embodiment is a system for nerve modulation, including an elongate shaft having a proximal end region and a distal end region. The system may further include a first inflatable balloon having a proximal end and a distal end, the first inflatable balloon disposed proximate the distal end region of the elongate shaft. An outer member having an inner surface and an outer surface may be disposed over the first inflatable balloon and extending from the proximal end to the distal end thereof. The system may further include a nerve modulation element disposed on the outer member. In some instances, the system may include a second inflatable balloon positioned approximately 180° from the first inflatable balloon on the elongate shaft. The system may further include more than one nerve ablation element. The outer member may include an open region defining a window. The nerve modulation element may be attached to the inner surface of the outer member such that at least a portion of the nerve modulation element is visible through the window. The outer member may further include a vent. In some embodiments, the vent may extend between the outer member and the elongate shaft. In other embodiments, the vent may extend between the inflatable balloon and the outer member.

Another illustrative embodiment is an intravascular nerve ablation system. The system may include an elongate shaft having a proximal end and distal end and a lumen extending therebetween. A first inflatable balloon and a second inflatable balloon having proximal ends and distal ends may be disposed at a first position and second position, respectively, proximate the distal end of the elongate shaft. A third balloon having an inner surface and an outer surface may be disposed over the first balloon and the second balloon. The third balloon may extend from the proximal ends of the first and second balloons to the distal ends of first and second balloons. A first opening may defined in a first side of the third balloon and a second opening may be defined in a second side of the third balloon, the openings extending from the inner surface to the outer surface. The system may further include a first electrode attached to the inner surface of the third balloon such that at least a portion of the electrode is visible through the first opening and a second electrode attached to the inner surface of the third balloon such that at least a portion of the electrode is visible through the second opening. The first position may be approximately 180° from the second position, the first opening approximately 180° from the second opening, and the first opening approximately 90° from the first position.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 illustrates a distal end of an illustrative renal nerve modulation system.

FIG. 3 is a cross-section of the illustrative renal nerve modulation system shown in FIG. 2.

FIG. 4 is an end view of the illustrative renal nerve modulation system shown in FIG. 2.

FIG. 5 illustrates a distal end of an illustrative renal nerve modulation system.

FIG. 6 is an end view of the illustrative renal nerve modulation system shown in FIG. 5.

FIG. 7 illustrates a distal end of an illustrative renal nerve modulation system.

FIG. 8 is an end view of the illustrative renal nerve modulation system shown in FIG. 7.

FIG. 9 illustrates a perspective view of a distal end of an illustrative renal nerve modulation system.

FIGS. 9A and 9B are cross-sections of the illustrative renal nerve modulation system shown in FIG. 9.

FIG. 10 illustrates a distal end of an illustrative renal nerve modulation system.

FIG. 11 is an end view of the illustrative renal nerve modulation system shown in FIG. 10.

FIG. 12 illustrates a distal end of an illustrative renal nerve modulation system.

FIG. 13 is an end view of the illustrative renal nerve modulation system shown in FIG. 12.

FIG. 14 illustrates a distal end of an illustrative renal nerve modulation system.

FIG. 15 is an end view of the illustrative renal nerve modulation system shown in FIG. 14.

FIG. 16 illustrates a distal end of an illustrative renal nerve modulation system.

FIG. 17 is an end view of the illustrative renal nerve modulation system shown in FIG. 16.

FIG. 18 illustrates a distal end of an illustrative renal nerve modulation system.

FIG. 19 is a cross-section of the illustrative renal nerve modulation system shown in FIG. 18.

FIGS. 20A-20C illustrate the cross-section shown in FIG. 19 in various circumferential positions.

FIG. 21 illustrates a distal end of an illustrative renal nerve modulation system.

FIG. 22 is a cross-section of the illustrative renal nerve modulation system shown in FIG. 21.

FIG. 23 illustrates a distal end of an illustrative renal nerve modulation system.

FIG. 24 is an end view of the illustrative renal nerve modulation system shown in FIG. 23.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation, it is contemplated that the devices and methods may be used in other applications where nerve modulation and/or ablation are desired. In some instances, it may be desirable to ablate perivascular renal nerves with deep target tissue heating. However, as energy passes from an electrode to the desired treatment region the energy may heat the fluid (e.g. blood) and tissue as it passes. As more energy is used, higher temperatures in the desired treatment region may be achieved thus resulting in a deeper lesion. However, this but may result in some negative side effects, such as, but not limited to thermal injury to the vessel wall, blood damage, clotting and/or protein fouling of the electrode. Positioning the electrode away from the vessel wall may provide some degree of passive cooling by allowing blood to flow past the electrode. However, it may be desirable to provide an increased level of cooling over the passive cooling generated by normal blood flow. In some instances, a partial occlusion catheter may be used to partially occlude an artery or vessel during nerve ablation. The partial occlusion catheter may reduce the cross-sectional area of the vessel available for blood flow which may increase the velocity of blood flow in a region proximate the desired treatment area while minimally affecting the volume of blood passing, if at all. The increased velocity of blood flow may increase the convective cooling of the blood and tissues surrounding the treatment area and reducing artery wall thermal injury, blood damage, and/or clotting. The increased velocity of blood flow may also reduce protein fouling of the electrode. The renal nerve modulation systems described herein may include other mechanisms to improve convective heat transfer, such as, but not limited to directing flow patterns with surfaces, flushing fluid from a guide catheter or other lumen, or infusing cool fluid.

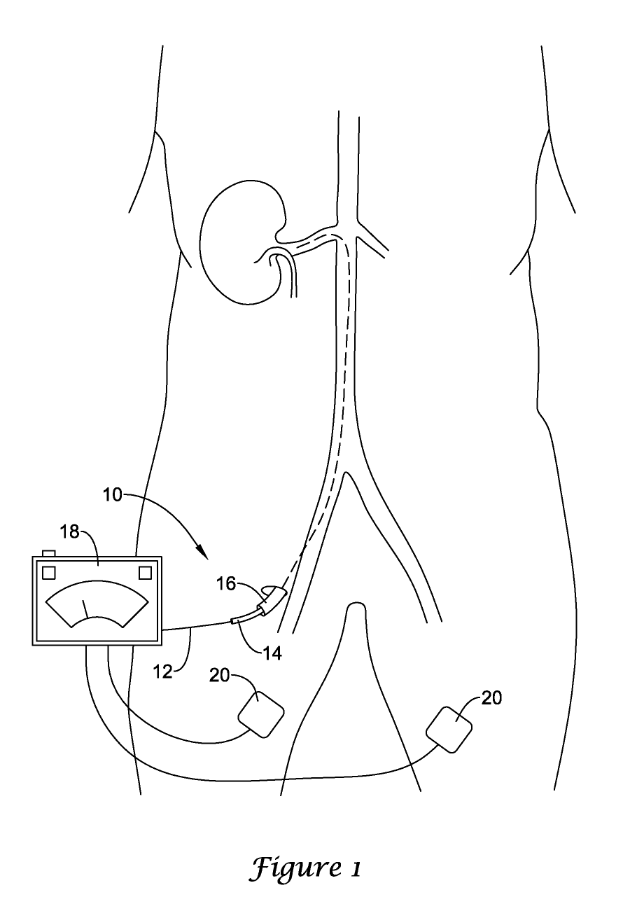

FIG. 1 is a schematic view of an illustrative renal nerve modulation system 10 in situ. System 10 may include an element 12 for providing power to an electrode disposed about and/or within a central elongate shaft 14 and, optionally, within a sheath 16, the details of which can be better seen in subsequent figures. A proximal end of element 12 may be connected to a control and power element 18, which supplies the necessary electrical energy to activate the one or more electrodes at or near a distal end of the element 12. In some instances, ground electrodes or return electrode patches 20 may be supplied on the legs or at another conventional location on the patient's body to complete the circuit. The control and power element 18 may include monitoring elements to monitor parameters such as power, temperature, voltage, pulse size and/or shape and other suitable parameters as well as suitable controls for performing the desired procedure. In some instances, the power element 18 may control a radio frequency (RF) electrode. The electrode may be configured to operate at a frequency of approximately 460 kHz. It is contemplated that any desired frequency in the RF range may be used, for example, from 450-500 kHz. However, it is contemplated that different types of energy outside the RF spectrum may be used as desired, for example, but not limited to ultrasound, microwave, and laser.

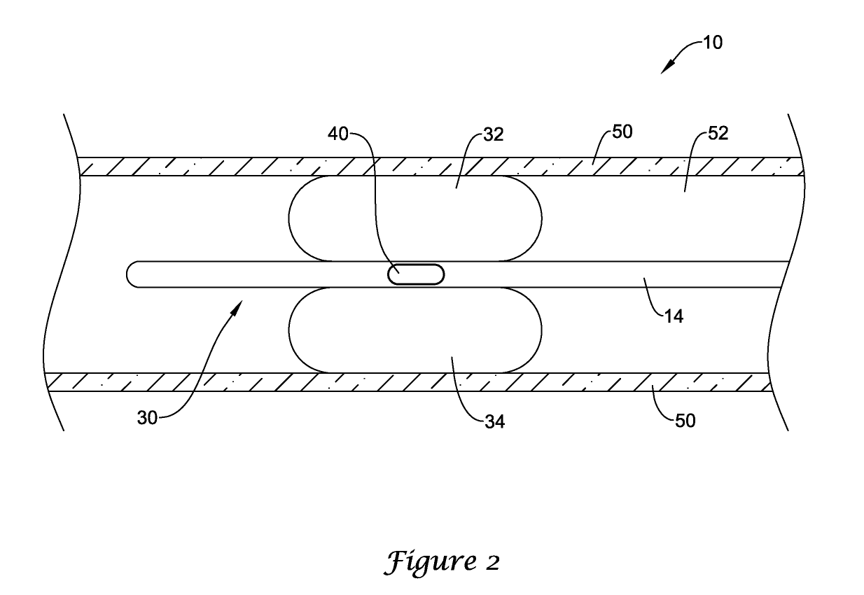

FIG. 2 is an illustrative embodiment of a distal end of a renal nerve modulation system 10 disposed within a body lumen 52 having a vessel wall 50. The system 10 may include an elongate shaft 14 having a distal end region 30. The elongate shaft 14 may extend proximally from the distal end region 30 to a proximal end configured to remain outside of a patient's body. The proximal end of the elongate shaft 14 may include a hub attached thereto for connecting other treatment devices or providing a port for facilitating other treatments. The elongate shaft 14 may further include one or more lumens extending therethrough. For example, the elongate shaft 14 may include a guidewire lumen and/or one or more inflation lumens. The lumens may be configured in any way known in the art. For example, the guidewire lumen may extend the entire length of the elongate shaft 14 such as in an over-the-wire catheter or may extend only along a distal portion of the elongate shaft 14 such as in a single operator exchange (SOE) catheter. These examples are not intended to be limiting, but rather examples of some possible configurations. While not explicitly shown, the modulation system 10 may further include temperature sensors/wire, an infusion lumen, radiopaque marker bands, fixed guidewire tip, a guidewire lumen, external sheath and/or other components to facilitate the use and advancement of the system 10 within the vasculature may be incorporated.

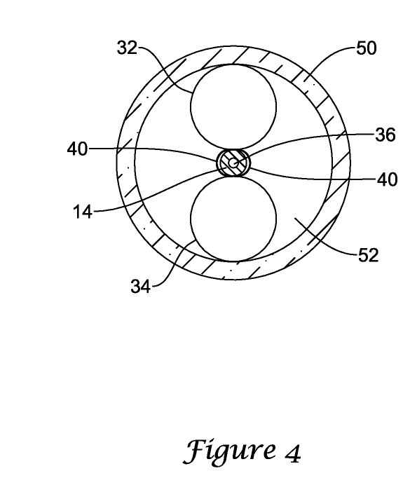

The modulation system 10 may include a first inflatable balloon 32 and a second inflatable balloon 34 disposed on or adjacent to the elongate shaft 14 at the distal end region 30. In some instances, the first and second balloons 32, 34 may be positioned on the distal end region 30 of the elongate shaft 14 approximately 180° from one another. However, the balloons 32, 34 may have any radial or circumferential arrangement desired. While the balloons 32, 34 are shown as having a circular cross-section (see FIG. 4), it is contemplated the balloons 32, 34 may have any shape or size desired. In some embodiments, the first and second balloons 32, 34 may be secured directly to the elongate shaft 14 in any manner desired. In other embodiments, the first and second balloons 32, 34 may be secured to the elongate shaft 14 in such a way that the balloons 32, 34 do not directly contact the elongate shaft 14. It is contemplated that the stiffness of the elongate shaft 14 in combination with the compliance of the balloon(s) 32, 34 may be modified to form modulations systems 10 for use in various vessel diameters. The balloons discussed herein, in this embodiment and in the preceding and following embodiments, are generally made from an insulating material or from a material that does not conduct electricity well, except as otherwise specifically described. Thus, current density travelling between one electrode and another or between one electrode and a ground will avoid travelling through the balloon material.

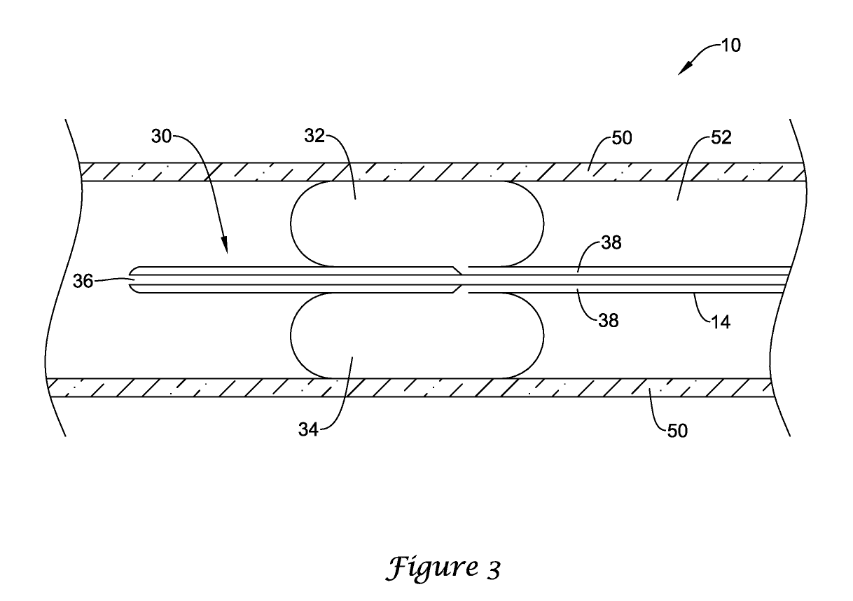

FIG. 3 illustrates a longitudinal cross-section of the illustrative ablation system of FIG. 2. First and second balloons 32, 34 may be fluidly connected to an inflation lumen 38 disposed within the elongate shaft 14 such that the balloons 32, 34 may be inflated and deflated. While the balloons 32, 34 are shown in direct contact with the elongate shaft 14, it is contemplated that the balloons 32, 34 and inflation lumen 38 may have any configuration desired.

The modulation system 10 may be advanced through the vasculature in any manner known in the art. For example, system 10 may include a guidewire lumen 36 to allow the system 10 to be advanced over a previously located guidewire. In some embodiments, the modulation system 10 may be advanced, or partially advanced, within a guide sheath such as the sheath 16 shown in FIG. 1. The first and second balloons 32, 34 may be deflated during introduction, advancement, and removal of the system 10. Once the distal end region 30 of the modulation system 10 has been placed adjacent to the desired treatment area, the balloons 32, 34 may be inflated to partially occlude the vessel lumen 52. Once inflated the balloons 32, 34 may reduce the cross-sectional area of the vessel and may maintain consistent spacing between the vessel wall 50 and the electrode 40. The inflated balloons 32, 34 may occupy to 50% or more of the vessel lumen 52 (cross-section) over a short distance (approximately 1-2 cm) without significantly affecting the volumetric flow of blood capable of passing the partial occlusion. The partial occlusion of the lumen 52 may increase the flow rate (velocity) of blood through the remaining portion of the lumen 52 which may result in an increased amount of convective cooling in the treatment region. It is further contemplated that the balloons 32, 34 may be deflated at the treatment region to allow for longitudinal and radial adjustment of the modulation system 10. For example, in some instances, the modulation system 10 may be energized several different times while the elongate shaft 14 is longitudinally displaced in order to perform an ablation over a desired length. It is contemplated that in some embodiments, the system 10 may include electrodes 40 positioned at various positions along the length of the modulation system 10 such that a larger region may be treated without longitudinal displacement of the elongate shaft 14. Further, in some instances, such as when an electrode 40 does not extend around the entire perimeter of the elongate shaft 14, the shaft 14 may need to be rotated 90° to complete the ablation process.

Returning to FIG. 2, the system 10 may further include one or more electrodes 40 disposed on the outer surface of the elongate shaft 14. In some instances the one or more electrodes 40 may be positioned between the first and second balloons 32, 34, as shown more clearly in FIG. 4. In some embodiments, the electrode(s) 40 may be formed of a separate structure and attached to the elongate shaft 14. For example, the electrode(s) 40 may be machined or stamped from a monolithic piece of material and subsequently bonded or otherwise attached to the elongate shaft 14. In other embodiments, the electrode(s) 40 may be formed directly on the surface of the elongate shaft 14. For example, the electrode(s) 40 may be plated, printed, or otherwise deposited on the surface. In some instances, the electrode(s) 40 may be radiopaque marker bands. The electrode(s) 40 may be formed from any suitable material such as, but not limited to, platinum, gold, stainless steel, cobalt alloys, or other non-oxidizing materials. In some instances, titanium, tantalum, or tungsten may be used. It is contemplated that the electrode(s) 40 may take any shape desired, such as, but not limited to, square, rectangular, circular, oblong, etc. In some embodiments, the electrode(s) 40 may have rounded edges in order to reduce the affects of sharp edges on current density. The size of the electrode(s) 40 may be chosen to optimize the current density without increasing the profile of the modulation system 10. For example, an electrode 40 that is too small may generate high local current densities resulting in greater heat transfer to the blood and surrounding tissues. An electrode 40 that is too large may require a larger elongate shaft 14 to carry it. It is contemplated that with a suitably flexible material, electrodes 40 of any size may be placed on one or both of the balloons 32, 34. In some instances, the electrode(s) 40 may have an aspect ratio of 2:1 (length to width). Such an elongated structure may provide the electrode(s) 40 with more surface area without increasing the profile of the modulation system 10. While the electrode(s) 40 are shown as disposed on the elongate shaft 14, it is contemplated that in some embodiments, the electrode(s) 40 may be disposed on the surface of one, or both, of the balloons 32, 34. In other embodiments, a region of one, or both, of the balloons 32, 34 may be made conductive. In some embodiments, the electrodes 40 may be a single electrode disposed around the entire perimeter of the elongate shaft 14. A single electrode 40 may allow for 360° ablation. Thus, the elongate shaft 14 may not require circumferential repositioning.

FIG. 4 illustrates an end view of the illustrative modulation system 10 of FIG. 2 disposed within a body lumen 52. While the system 10 is illustrated as including two electrodes 40, it is contemplated the system may include any number of electrodes 40 desired, for example one, two, three, four, or more. In some instances, the electrodes 40 may be positioned on the distal end region 30 of the elongate shaft 14 approximately 180° from one another. However, the electrodes 40 may be positioned in any radial or circumferential position desired. Further, in some embodiments, electrodes 40 may be placed at different longitudinal positions along the length of the elongate shaft 14.

The balloons 32, 34 may space the electrodes 40 a distance from the vessel wall 50 in an off-the-wall or non-contact arrangement. The balloons 32, 34 may further maintain consistent spacing between the vessel wall 50 and the electrodes 40 such that fluid flow past the electrodes 40 may be preserved. However, in some embodiments, the balloons 32, 34 and/or elongate shaft 14 may be arranged such that the electrodes 40 contact the vessel wall 50. While not explicitly shown, the electrodes 40 may be connected to a control unit (such as control unit 18 in FIG. 1) by electrical conductors. Once the modulation system 10 has been advanced to the treatment region, energy may be supplied to the electrodes 40. The amount of energy delivered to the electrodes 40 may be determined by the desired treatment. For example, more energy may result in a larger, deeper lesion. In some embodiments, it may be desired to achieve the hottest, deepest lesion beyond the vessel wall 50 while minimizing the temperature at the surface of the vessel wall 50. The temperature at the surface of the vessel wall 50 may be a function of the power used as well as the fluid flow through the body lumen 52. In some instances, the increased velocity of fluid flow resulting from the partial vessel occlusion may allow more power to be used during treatment. While the current density traveling between, for example, electrode 40 and ground electrode 20 (shown in FIG. 1) may result in the heating of adjacent fluid and tissue, there may be negligible resistance in the electrode 40 such that the electrode 40 does not get hot.

It is contemplated that the modulation system 10 may be operated in a variety of modes. In one embodiment, the system 10 may be operated in a sequential unipolar ablation mode. For example, the distal end region 30 including the balloons 32, 34 may bisect the vessel lumen 52 with an electrode 40 on either side (as shown in FIG. 4), but this is not required. The electrodes 40 may each be connected to an independent power supply such that each electrode 40 may be operated separately and current may be maintained to each electrode 40. In sequential unipolar ablation, one electrode 40 may be activated such that the current travels from the electrode 40 between the balloons 32, 34 to the ground electrode 20. Once one side has been ablated, the other electrode 40 may be activated such that current travels from the electrode 40 between the balloons 32, 34 to the ground electrode 20 and ablating the other side.

In another embodiment, the system 10 may be operated in a simultaneous unipolar ablation mode. Similar to the sequential unipolar mode, the simultaneous unipolar mode the distal end region 30 including the balloons 32, 34 may bisect the vessel lumen 52 with an electrode 40 on either side (as shown in FIG. 4), but this is not required. In simultaneous unipolar ablation mode, both electrodes 40 may be activated simultaneously such that current travels from each electrode 40 between the balloons 32, 34 to the ground electrodes 20. In some instances, the electrodes 40 may each be connected to an independent electrical supply such that current is maintained to each electrode 40. In this mode, more current may be dispersed circumferentially. This may result in a more effective, deeper penetration compared to the sequential unipolar ablation mode.

In another embodiment, the system 10 may be operated in a bipolar mode. In this instance, two electrodes 40 disposed at the treatment location may be 180° out of phase such that one electrode 40 acts as the ground electrode (e.g. one cathode and one anode). As such current may flow around the elongate shaft 14 and around balloons 32, 34 from one electrode 40 to the other electrode 40. In general, either sequential or simultaneous unipolar mode may penetrate more deeply than the bipolar mode. Because balloons 32, 34 are generally insulating, the current density is forced around the balloons, and thus more of the current density penetrates the vessel wall 50 and surrounding tissue. While described with respect to the illustrative embodiment of FIGS. 2-4 it is to be understood that any of the embodiments described herein may be operated in any of the above described modes.

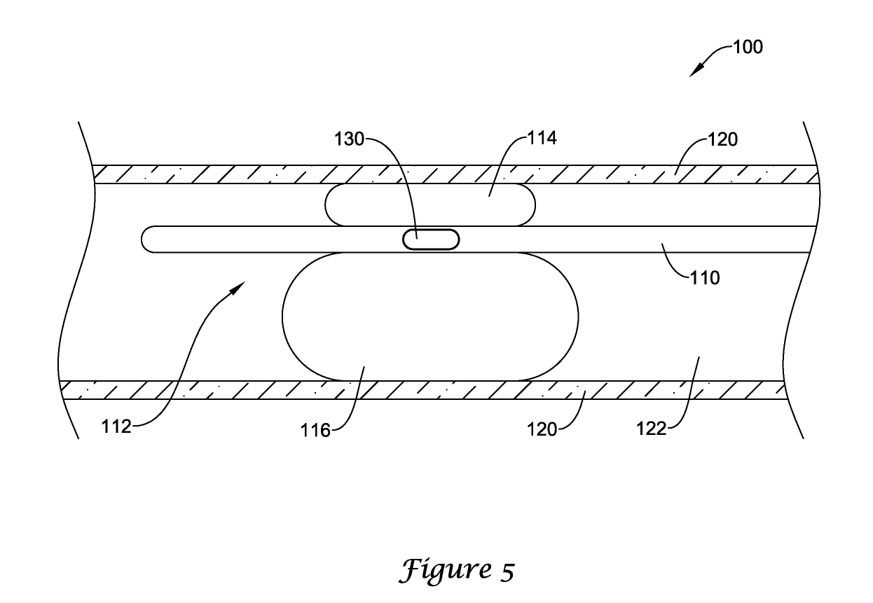

FIG. 5 is another illustrative embodiment of a distal end of a renal nerve modulation system 100 disposed within a body lumen 122 having a vessel wall 120. The system 100 may include an elongate shaft 110 having a distal end region 112. The system 100 may include a first inflatable balloon 114 and a second inflatable balloon 116 disposed on or adjacent to the elongate shaft 110. As illustrated, the first balloon 114 may be smaller than the second balloon 116. This may allow the electrode 130 to be positioned closer to one side of the vessel wall 120. Further, such an arrangement may block a greater portion of the vessel lumen 122 resulting in an even greater increase in velocity and hence convective cooling. In some instances, the first and second balloons 114, 116 may be positioned on the distal end region 112 of the elongate shaft 110 approximately 180° from one another. However, it is contemplated the balloons 114, 116 may have any radial or circumferential arrangement desired. In some embodiments, the first and second balloons 114, 116 may be secured directly to the elongate shaft 110 in any manner desired. In other embodiments, the first and second balloons 114, 116 may be secured to the elongate shaft 110 in such a way that the balloons 114, 116 do not directly contact the elongate shaft 110. It is contemplated that the balloons 114, 116 may be connected and operated as discussed with respect to the balloons 32, 34 illustrated in FIGS. 2-4. It is further contemplated that the system 100 and elongate shaft 110 may incorporate the features and may use the methods described with respect to the modulation system 10 and elongate shaft 14 illustrated in FIGS. 2-4.

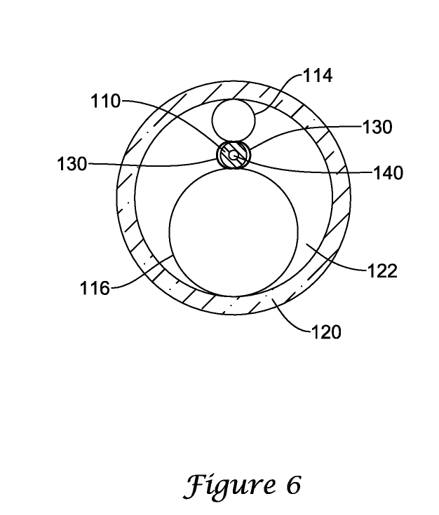

The system 100 may further include one or more electrodes 130 disposed on the outer surface of the elongate shaft 110. In some instances the one or more electrodes 130 may be positioned between the first and second balloons 114, 116, as shown more clearly in FIG. 6. The electrode(s) 130 may be formed and attached to the shaft 110 in the manner described with respect to electrodes 40 shown in FIGS. 2-4. The electrode(s) 130 may be formed of any suitable material, shape, and size such as those described with respect to electrodes 40 shown in FIGS. 2-4. While the electrode(s) 130 are shown as disposed on the elongate shaft 110, it is contemplated that in some embodiments, the electrode(s) 130 may be disposed on the surface of one, or both, of the balloons 114, 116. In other embodiments, a region of one, or both, of the balloons 114, 116 may be made conductive.

FIG. 6 illustrates an end view of the illustrative modulation system 100 of FIG. 5 disposed within a vessel lumen 122. In some instances, the elongate shaft 110 may include a lumen 140 for receiving a guidewire or other device. While the system 100 is illustrated as including two electrodes 130, it is contemplated the system may include any number of electrodes 130 desired, for example one, two, three, four, or more. In some instances, the electrodes 130 may be positioned on the distal end region 112 of the elongate shaft 110 approximately 180° from one another. However, the electrodes 130 may be positioned in any radial or circumferential position desired. Further, in some embodiments, electrodes 130 may be placed at different longitudinal positions along the length of the elongate shaft 110.

The balloons 114, 116 may space the electrodes 130 a distance from the vessel wall 120 in an off-the-wall or non-contact arrangement. The balloons 114, 116 may further maintain consistent spacing between the vessel wall 120 and the electrodes 130 such that fluid flow past the electrodes 130 may be preserved. As can be seen, the first balloon 114 may have a smaller cross-section than the second balloon 116. Thus, the electrodes 130 may be positioned closer to one side of the vessel wall 120.

While not explicitly shown, the electrodes 130 may be connected to a control unit (such as control unit 18 in FIG. 1) by electrical conductors. Once the modulation system 100 has been advanced to the treatment region, energy may be supplied to the electrodes 130. The amount of energy delivered to the electrodes 130 may be determined by the desired treatment. For example, more energy may result in a larger, deeper lesion. In some embodiments, it may be desired to achieve the hottest, deepest lesion beyond the vessel wall while minimizing the temperature at the surface of the vessel wall 120. The temperature at the surface of the vessel wall 120 may be a function of the power used as well as the fluid flow through the vessel lumen 122. In some instances, the increased velocity of fluid flow resulting from the partial vessel occlusion may allow more power to be used during treatment. While the current density traveling between, for example, electrode 130 and ground electrode 20 (shown in FIG. 1) may result in the heating of adjacent fluid and tissue, there may be negligible resistance in the electrode 130 such that the electrode 130 does not get hot.

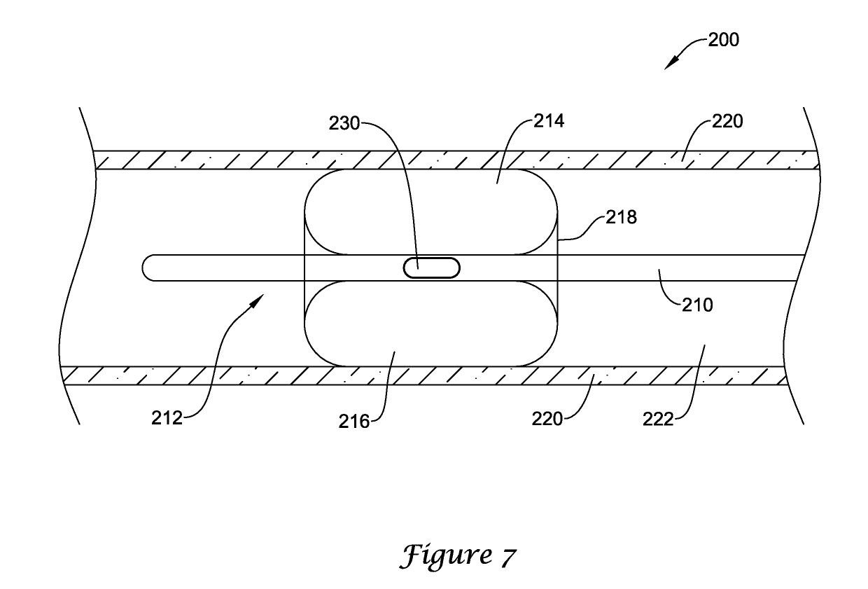

FIG. 7 is another illustrative embodiment of a distal end of a renal nerve modulation system 200 disposed within a body lumen 222 having a vessel wall 220. The system 200 may include an elongate shaft 210 having a distal end region 212. It is contemplated that the system 200 and elongate shaft 210 may incorporate the features and may use the methods described with respect to the modulation system 10 and elongate shaft 14 illustrated in FIGS. 2-4. The system 200 may include a first inflatable balloon 214 and a second inflatable balloon 216 disposed on or adjacent to the elongate shaft 210. In some instances, the first and second balloons 214, 216 may be positioned on the distal end region 212 of the elongate shaft 210 approximately 180° from one another. However, it is contemplated the balloons 214, 216 may have any radial or circumferential arrangement desired. In some embodiments, the first and second balloons 214, 216 may be secured directly to the elongate shaft 210 in any manner desired. In other embodiments, the first and second balloons 214, 216 may be secured to the elongate shaft 210 in such a way that the balloons 214, 216 do not directly contact the elongate shaft 210. It is contemplated that the balloons 214, 216 may be connected and operated as discussed with respect to the balloons 32, 34 illustrated in FIGS. 2-4.

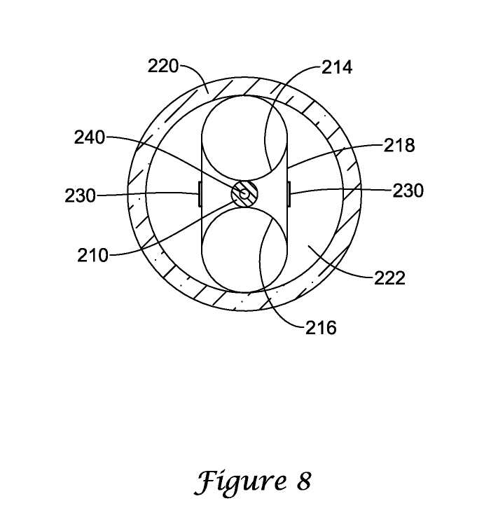

The modulation system 200 may further include an outer member 218, such as an outer sheath or balloon disposed over the first and second balloons 214, 216. The outer balloon 218 may extend from a proximal end of the first and second balloons 214, 216 to a distal end of the first and second balloons 214, 216. When the first and second balloons 214, 216 are expanded, the outer balloon 218 may have an oblong shape. As shown in FIG. 8, the outer balloon 218 may further occlude the vessel lumen 222 and provide greater convective cooling than with the first and second balloons 214, 216 alone.

The system 200 may further include one or more electrode(s) 230 disposed on the outer surface of the outer balloon 218. The electrode(s) 230 may be supported by a strut or other supporting means. The electrode(s) 230 may be formed and attached to the outer balloon 218 in the manner described with respect to electrodes 40 shown in FIGS. 2-4. In some embodiments, a window may be formed in the outer balloon 218 (for example, a section of the balloon 218 may be removed) and the electrode(s) 230 may be attached to an inner surface of the outer balloon 218 such that portion of the electrode 230 is exposed through the window. This may allow the edges of the electrode 230 to be insulated, thus reducing high local current densities. The electrode(s) 230 may be formed of any suitable material, shape, and size such as those described with respect to electrodes 40 shown in FIGS. 2-4. While the electrode(s) 230 are shown as disposed on the outer balloon 218, it is contemplated that in some embodiments, the electrode(s) 230 may be disposed on the surface of the elongate shaft 210 or one, or both, of the balloons 214, 216. In other embodiments, a region of the outer balloon 218 or one, or both, of the balloons 214, 216 may be made conductive.

In some embodiments, the outer balloon 218 may not be fluidly connected to an inflation lumen. The outer balloon 218 may expand and contract as the first and second balloons 214, 216 are inflated and deflated. However, in some embodiments, the outer balloon 218 may be fluidly connected to an inflation lumen such that the outer balloon 218 may be expanded and contracted independently of the first and second balloons 214, 216. While not explicitly shown, the outer balloon 218 may include a vent to allow fluid to enter and exit the outer balloon 218. This may be accomplished, for example, through an inflation lumen within the elongate shaft 210. Alternatively, microscopic openings may be disposed in the surfaces of the first, second, and outer balloons 214, 216, 218. This may allow for a controlled “leak” of inflation fluid to transfer from the first and second balloons 214, 216 to the outer balloon 218 and finally into the vessel lumen 222. This may help prevent a vacuum from forming.

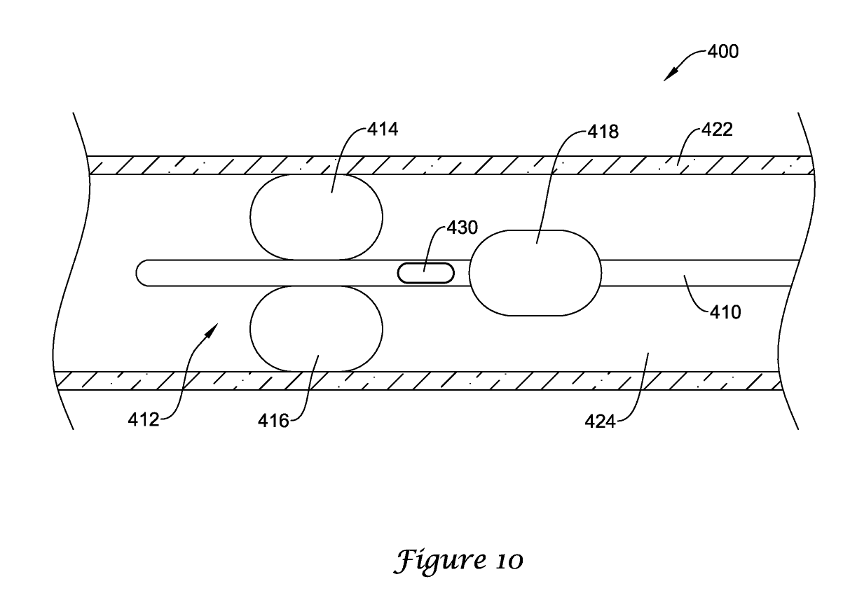

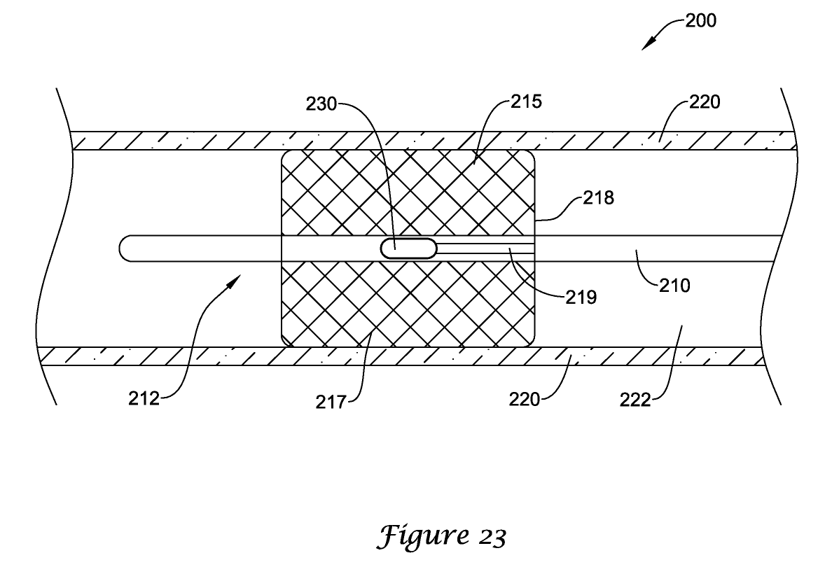

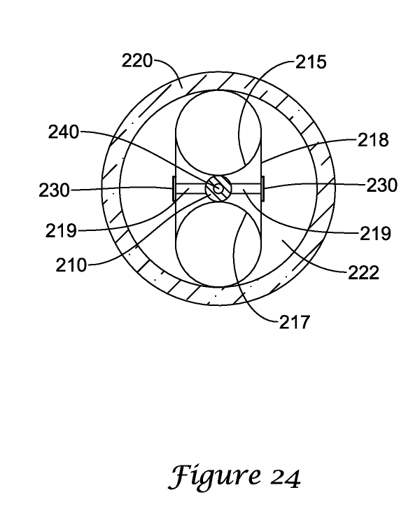

In other embodiments, a self-expanding stent may be used in place of the first and second balloons 214, 216. For example, the stent may include a covered stent or a slotted tube, among other structures. A self-expanding stent may provide more robust support for the electrode(s) 230. FIGS. 23 and 24 illustrate an example of such a system. In this system, similar to that described with respect to FIGS. 7 and 8 in other respects, self-expanding cages 215 and 217 are used to expand outer balloon 218. These self-expanding cages 215, 217 may expand, for example, when a sheath (not illustrated) is withdrawn proximally from a restraining position over the distal end of the modulation system. In some embodiments, electrodes 230 are fixed to struts 219, which in turn are fixed to elongate shaft 210. The struts 219 are biased to the deployed position. When a sheath (not illustrated) is withdrawn proximally from the distal end of the modulation system, the struts can spring out to their illustrated deployed positions. In some embodiments, one or more of the electrodes 230 may be fixed directly to the self-expanding cages. While the use of self-expanding cages is illustrated, it is to be understood that any self-expanding structure, such as a slotted tube or stent may be used. Further, the use of self-expanding cages is not limited to the embodiment illustrated, and a self-expanding structure may readily be substituted for a balloon in any of the embodiments. Such structures may be bare or may be covered (e.g. have a fluid impermeable covering) on only their outer circumferential surface or may have their proximal or distal or both proximal and distal ends covered or any combination thereof. For example, in the embodiment of FIG. 7, a single self-expanding cage having the same profile as outer balloon 218 may be readily substituted for the pair of balloons 214, 216, and be used to expand balloon 218 to the shape illustrated. Similarly, covered self-expanding cages may be substituted, for example, for the balloons 414, 416, 418 in the embodiment of FIG. 10 described below.

FIG. 8 illustrates an end view of the illustrative modulation system 200 shown in FIG. 7 disposed within a vessel lumen 222. In some instances, the elongate shaft 210 may include a lumen 240 for receiving a guidewire or other device. While the system 200 is illustrated as including two electrodes 230, it is contemplated the system may include any number of electrodes 230 desired, for example one, two, three, four, or more. In some instances, the electrodes 230 may be positioned on the outer balloon 218 approximately 180° from one another. However, the electrodes 230 may be positioned in any radial or circumferential position desired. Further, in some embodiments, electrodes 230 may be placed at different longitudinal positions along the length of the outer balloon 218 and/or elongate shaft 210. The balloons 214, 216 may space the electrodes 230 a distance from the vessel wall 220 in an off-the-wall or non-contact arrangement. The balloons 214, 216 may further maintain consistent spacing between the vessel wall 220 and the electrodes 230 such that fluid flow past the electrodes 230 may be preserved. The outer balloon 218 may position the electrodes 230 closer to the vessel wall 220 than an embodiment where the electrodes are located on the elongate shaft.

While not explicitly shown, the electrodes 230 may be connected to a control unit (such as control unit 18 in FIG. 1) by electrical conductors. Once the modulation system 200 has been advanced to the treatment region, energy may be supplied to the electrodes 230. The amount of energy delivered to the electrodes 230 may be determined by the desired treatment. For example, more energy may result in a larger, deeper lesion. In some embodiments, it may be desired to achieve the hottest, deepest lesion beyond the vessel wall while minimizing the temperature at the surface of the vessel wall 220. The temperature at the surface of the vessel wall 220 may be a function of the power used as well as the fluid flow through the vessel lumen 222. In some instances, the increased velocity of fluid flow resulting from the partial vessel occlusion may allow more power to be used during treatment. While the current density traveling between, for example, electrode 230 and ground electrode 20 (shown in FIG. 1) may result in the heating of adjacent fluid and tissue, there may be negligible resistance in the electrode 230 such that the electrode 230 does not get hot.

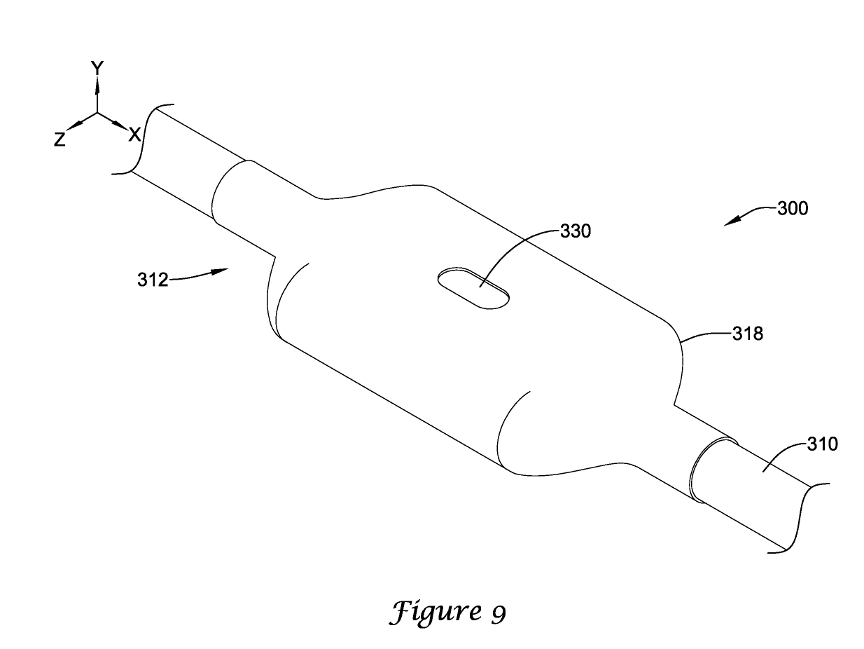

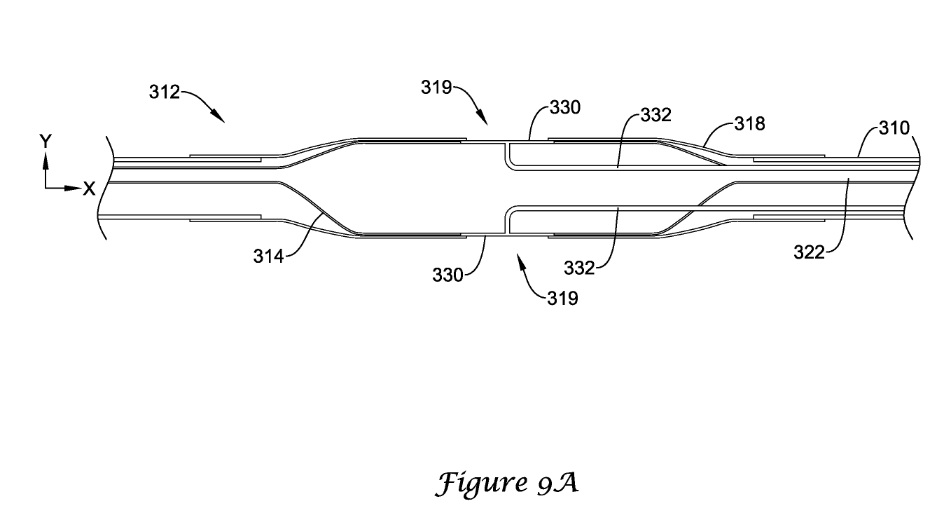

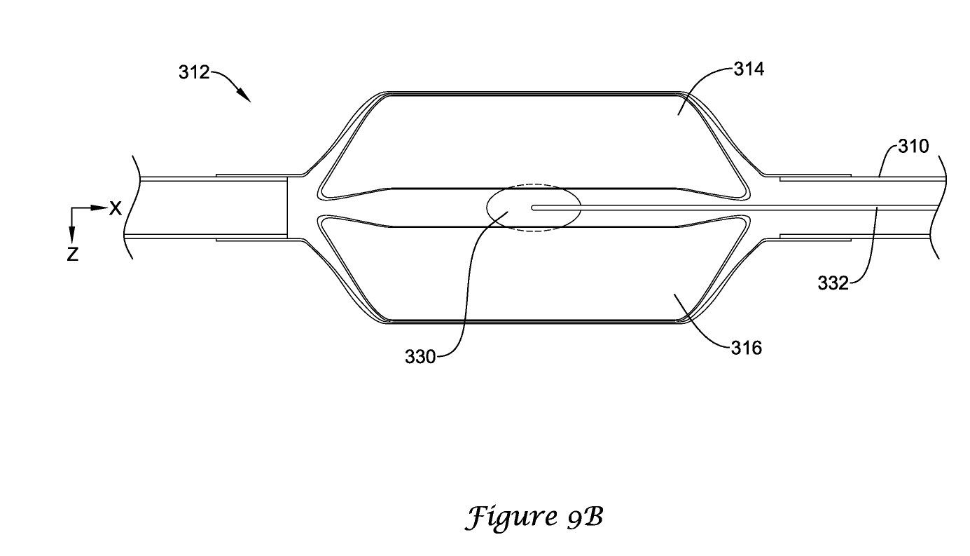

FIG. 9 is a perspective view of another illustrative embodiment of a distal end of a renal nerve modulation system 300. The system 300 may include an elongate shaft 310 having a distal end region 312. It is contemplated that the system 300 and elongate shaft 310 may incorporate the features and may use the methods described with respect to the modulation system 10 and elongate shaft 14 illustrated in FIGS. 2-4. The modulation system 300 may include an outer balloon 318 disposed over a first inner balloon 314 and a second inner balloon 316 (see FIG. 9B). The outer balloon 318 may be connected to the elongate shaft 310 in any manner desired. For example, the outer balloon 318 may heat shrunk, welded, bonded, thermally bonded, etc. to the elongate shaft 310. While not explicitly shown, the outer balloon 318 may include a vent to allow fluid to enter and exit the outer balloon 318. This may be accomplished, for example, through an inflation lumen within the elongate shaft 310. Alternatively, microscopic openings may be disposed in the surfaces of the first, second, and outer balloons 314, 316, 318. This may allow for a controlled “leak” of inflation fluid to transfer from the first and second balloons 314, 316 to the outer balloon 318 and finally into the vessel lumen. This may help prevent a vacuum from forming.

The modulation system 300 may further include one or more electrode(s) 330 disposed on the outer surface of the outer balloon 318. The electrode(s) 330 may be supported by a strut or other supporting means. The electrode(s) 330 may be formed and attached to the outer balloon 318 in the manner described with respect to electrodes 40 shown in FIGS. 2-4. The electrode(s) 330 may be formed of any suitable material, shape, and size such as those described with respect to electrodes 40 shown in FIGS. 2-4. While the electrode(s) 330 are shown as disposed on the outer balloon 318, it is contemplated that in some embodiments, the electrode(s) 330 may be disposed on the surface of the elongate shaft 310 or one, or both, of the balloons 314, 316. In other embodiments, a region of the outer balloon 318 or one, or both, of the balloons 314, 316 may be made conductive.

In some embodiments, the outer balloon 318 may not be fluidly connected to an inflation lumen. The outer balloon 318 may expand and contract as the first and second balloons 314, 316 are inflated and deflated. However, in some embodiments, the outer balloon 318 may be fluidly connected to an inflation lumen such that the outer balloon 318 may be expanded and contracted independently of the first and second balloons 314, 316. In some embodiments, a self-expanding stent may be used in place of the first and second balloons 314, 316. For example, the stent may include a covered stent or a slotted tube, among other structures. A self-expanding stent may provide more robust support for the electrode(s) 330.

FIG. 9A illustrates a cross-section of the illustrative embodiment of FIG. 9 taken along the X-Y plane. The electrodes 330 may be connected to electrical conductors 332 configured to supply energy to the electrodes 330. In some instances, the electrical conductors 332 may function as a strut or support for the electrodes 330. A portion of the outer balloon 318 may be removed to provide a window 319 for the electrodes 330. The electrodes 330 may be attached to an inner surface of the balloon 318 adjacent to the window 319. This arrangement may allow the edges of the electrode 330 to be insulated, thus reducing high local current densities.

FIG. 9B illustrates a cross-section of the illustrative embodiment of FIG. 9 taken along the X-Z plane. The system 300 may include a first inflatable balloon 314 and a second inflatable balloon 316 disposed on or adjacent to the elongate shaft 310. In some instances, the first and second balloons 314, 316 may be positioned on the distal end region 312 of the elongate shaft 310 approximately 180° from one another. However, it is contemplated the balloons 314, 316 may have any radial or circumferential arrangement desired. In some embodiments, the first and second balloons 314, 316 may be secured directly to the elongate shaft 310 in any manner desired. In other embodiments, the first and second balloons 314, 316 may be secured to the elongate shaft 310 in such a way that the balloons 314, 316 do not directly contact the elongate shaft 310. It is contemplated that the balloons 314, 316 may be connected and operated in the same manner as discussed with respect to the balloons 32, 34 illustrated in FIGS. 2-4. The first and second balloons 314, 316 may be in fluid communication with one or more inflation lumens 322 (see FIG. 9A) configured to supply the balloons 314, 316 with an inflation fluid.

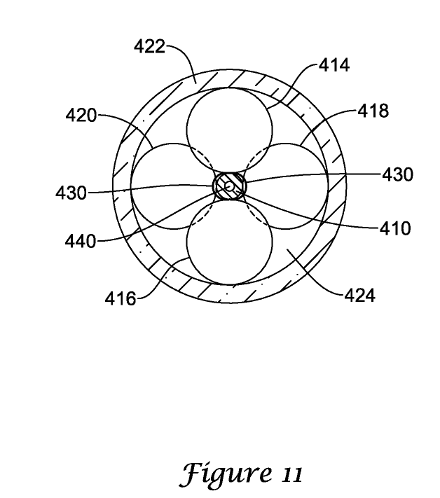

FIG. 10 is another illustrative embodiment of a distal end of a renal nerve modulation system 400 disposed within a body lumen 424 having a vessel wall 422. The system 400 may include an elongate shaft 410 having a distal end region 412. It is contemplated that the system 400 and elongate shaft 410 may incorporate the features and may use the methods described with respect to the modulation system 10 and elongate shaft 14 illustrated in FIGS. 2-4. The modulation system 400 may include inflatable balloons 414, 416, 418, 420 (see FIG. 11) at multiple locations along the length of the elongate shaft 410. In some instances, the balloons 414, 416, 418, 420 may be located on either side of the electrode 430 location. The system 400 may include a first inflatable balloon 414 and a second inflatable balloon 416 disposed on or adjacent to the elongate shaft 410 at location distal to the electrode 430. The system 400 may include a third inflatable balloon 418 and a fourth inflatable balloon 420 (shown in FIG. 11) disposed on or adjacent to the elongate shaft 410 at a location proximal to the electrode 430. It is contemplated that in some embodiments, the balloons 414, 416, 418, 420 may all be proximal or distal to the electrode 430.

In some instances, the first and second balloons 414, 416 may be positioned on the distal end region 412 of the elongate shaft 410 approximately 180° from one another. The third and fourth balloons 418, 420 may also be positioned on the distal end region 412 of the elongate shaft 410 approximately 180° from one another. However, it is contemplated the balloons 414, 416, 418, 420 may have any radial or circumferential arrangement desired. In some embodiments, the third and fourth balloons 418, 420 may be offset approximately 90° from the first and second balloons 414,416. However, in some embodiments, the third and fourth balloons 418, 420 may be aligned with the first and second balloons 414, 416. The arrangement of balloons at multiple locations along the elongate shaft 410 may provide improved centering and position without using longer balloons. Longer balloons may require an extended inflation/deflation time and create a more significant stiff region. Further, offset balloons may provide better positioning in multiple planes. Additionally, offset balloons may provide swirl or disturbed (more turbulence) flow for increased convective cooling. In some embodiments, the balloons 414, 416, 418, 420 may be secured directly to the elongate shaft 410 in any manner desired. In other embodiments, the balloons 414, 416, 418, 420 may be secured to the elongate shaft 410 in such a way that the balloons 414, 416, 418, 420 do not directly contact the elongate shaft 410. It is contemplated that the balloons 414, 416 may be connected and operated as discussed with respect to the balloons 32, 34 illustrated in FIGS. 2-4.

The system 400 may further include one or more electrodes 430 disposed on the outer surface of the elongate shaft 410. In some instances the one or more electrodes 430 may be positioned between the first and second balloons 414, 416 and the third and fourth balloons 418, 420. The electrode(s) 430 may be formed and attached to the shaft 410 in the manner described with respect to electrodes 40 shown in FIGS. 2-4. The electrode(s) 430 may be formed of any suitable material, shape, and size such as those described with respect to electrodes 40 shown in FIGS. 2-4. While the electrode(s) 430 are shown as disposed on the elongate shaft 410, it is contemplated that in some embodiments, the electrode(s) 430 may be disposed on the surface of one, or both, of the balloons 414, 416. In other embodiments, a region of one, or both, of the balloons 414, 416 may be made conductive. In some embodiments, the electrodes 430 may be a single electrode disposed around the entire perimeter of the elongate shaft 410. A single electrode 430 may allow for 360° ablation. Thus, the elongate shaft 410 may not require repositioning.

FIG. 11 illustrates an end view of the illustrative modulation system 400 shown in FIG. 10 disposed within a vessel lumen 424. In some instances, the elongate shaft 410 may include a lumen 440 for receiving a guidewire or other device. While the system 400 is illustrated as including two electrodes 430, it is contemplated the system may include any number of electrodes 430 desired, for example one, two, three, four, or more. In some instances, the electrodes 430 may be positioned on the distal end region 412 of the elongate shaft 410 approximately 180° from one another. However, the electrodes 430 may be positioned in any radial or circumferential position desired. Further, in some embodiments, electrodes 430 may be placed at different longitudinal positions along the length of the elongate shaft 410. The balloons 414, 416, 418, 420 may space the electrodes 430 a distance from the vessel wall 422 in an off-the-wall or non-contact arrangement. The balloons 414, 416, 418, 420 may further maintain consistent spacing between the vessel wall 422 and the electrode 430 such that fluid flow past the electrodes 430 may be preserved.

While not explicitly shown, the electrodes 430 may be connected to a control unit (such as control unit 18 in FIG. 1) by electrical conductors. Once the modulation system 400 has been advanced to the treatment region, energy may be supplied to the electrodes 430. The amount of energy delivered to the electrodes 430 may be determined by the desired treatment. For example, more energy may result in a larger, deeper lesion. In some embodiments, it may be desired to achieve the hottest, deepest lesion beyond the vessel wall while minimizing the temperature at the surface of the vessel wall 422. The temperature at the surface of the vessel wall 422 may be a function of the power used as well as the fluid flow through the vessel lumen 424. In some instances, the increased velocity of fluid flow resulting from the partial vessel occlusion may allow more power to be used during treatment. While the current density traveling between, for example, electrode 430 and ground electrode 20 (shown in FIG. 1) may result in the heating of adjacent fluid and tissue, there may be negligible resistance in the electrodes 430 such that the electrodes 430 do not get hot.

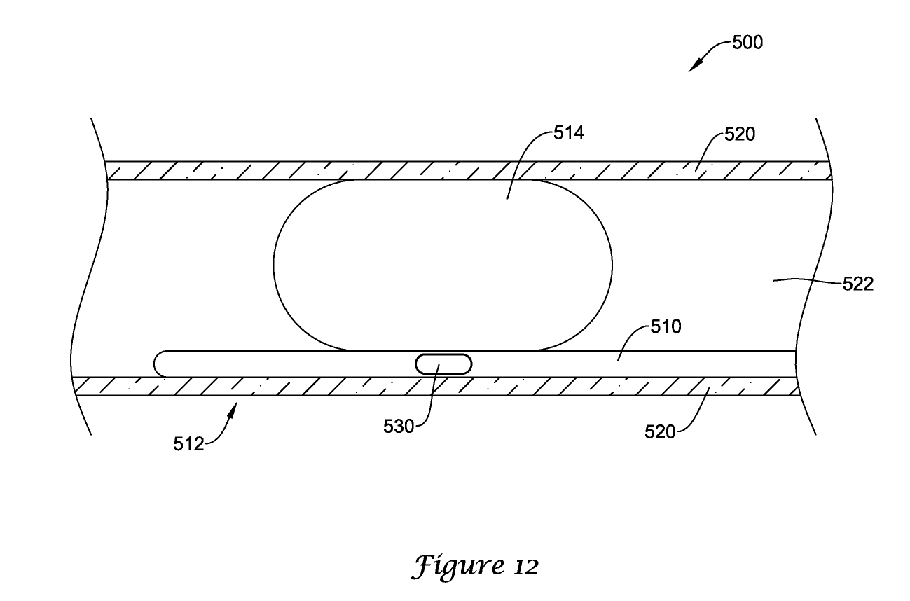

FIG. 12 is another illustrative embodiment of a distal end of a renal nerve modulation system 500 disposed within a body lumen 522 having a vessel wall 520. The system 500 may include an elongate shaft 510 having a distal end region 512. It is contemplated that the system 500 and elongate shaft 510 may incorporate the features and may use the methods described with respect to the modulation system 10 and elongate shaft 14 illustrated in FIGS. 2-4. The system 500 may include an inflatable balloon 514 disposed on or adjacent to the elongate shaft 510. In some embodiments, the balloon 514 may be secured directly to the elongate shaft 510 in any manner desired. In other embodiments, the balloon 514 may be secured to the elongate shaft 510 in such a way that the balloon 514 does not directly contact the elongate shaft 510. It is contemplated that the balloon 514 may be connected and operated as discussed with respect to the balloons 32, 34 illustrated in FIGS. 2-4. The balloon 514 may be sized and shaped to occlude a desired portion of the lumen 522.

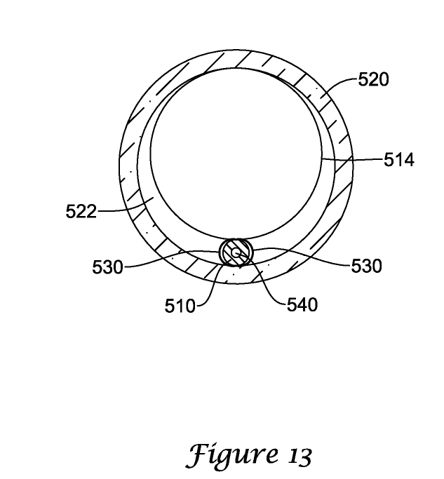

The system 500 may further include one or more electrodes 530 disposed on the outer surface of the elongate shaft 510. In some instances the one or more electrodes 530 may be positioned such that they are not in direct contact with the vessel wall 520 as shown more clearly in FIG. 13. The electrode(s) 530 may be formed and attached to the shaft 510 in the manner described with respect to electrodes 40 shown in FIGS. 2-4. The electrode(s) 530 may be formed of any suitable material, shape, and size such as those described with respect to electrodes 40 shown in FIGS. 2-4. While the electrode(s) 530 are shown as disposed on the elongate shaft 510, it is contemplated that in some embodiments, the electrode(s) 530 may be disposed on the surface of the balloon 514. In other embodiments, a region of the balloon 514 may be made conductive.

FIG. 13 illustrates an end view of the illustrative modulation system 500 shown in FIG. 12 disposed within a vessel 520. In some instances, the elongate shaft 510 may include a lumen 540 for receiving a guidewire or other device. While the system 500 is illustrated as including two electrodes 530, it is contemplated the system may include any number of electrodes 530 desired, for example one, two, three, four, or more. In some instances, the electrodes 530 may be positioned on the distal end region 512 of the elongate shaft 510 approximately 180° from one another. However, the electrodes 530 may be positioned in any radial or circumferential position desired. Further, in some embodiments, electrodes 530 may be placed at different longitudinal positions along the length of the elongate shaft 510. The balloon 514 may be sized and shaped to space the electrodes 530 a distance from the vessel wall 520 in an off-the-wall or non-contact arrangement. The balloon 514 may further maintain consistent spacing between the vessel wall 520 and the electrodes 530 such that fluid flow past the electrodes 530 may be preserved. The single balloon 514 may position the elongate shaft 510 close to the vessel wall 520 such that the electrodes 530 are positioned closer to one side of the vessel 520.

While not explicitly shown, the electrodes 530 may be connected to a control unit (such as control unit 18 in FIG. 1) by electrical conductors. Once the modulation system 500 has been advanced to the treatment region, energy may be supplied to the electrodes 530. The amount of energy delivered to the electrodes 530 may be determined by the desired treatment. For example, more energy may result in a larger, deeper lesion. In some embodiments, it may be desired to achieve the hottest, deepest lesion beyond the vessel wall while minimizing the temperature at the surface of the vessel wall 520. The temperature at the surface of the vessel wall 520 may be a function of the power used as well as the fluid flow through the vessel 520. In some instances, the increased velocity of fluid flow resulting from the partial vessel occlusion may allow more power to be used during treatment. While the current density traveling between, for example, electrode 530 and ground electrode 20 (shown in FIG. 1) may result in the heating of adjacent fluid and tissue, there may be negligible resistance in the electrode 530 such that the electrode 530 does not get hot.

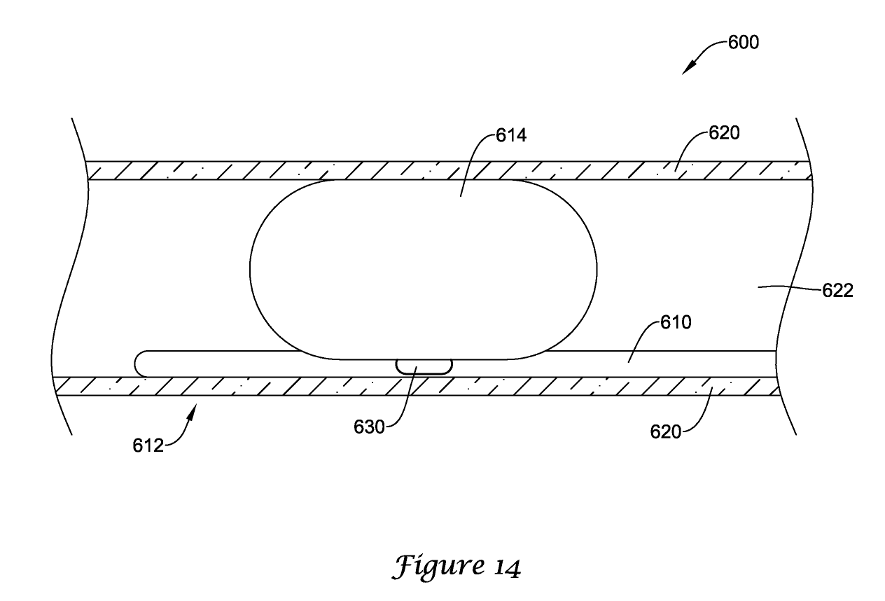

FIG. 14 is another illustrative embodiment of a distal end of a renal nerve modulation system 600 disposed within a body lumen 622 having a vessel wall 620. The system 600 may include an elongate shaft 610 having a distal end region 612. It is contemplated that the system 600 and elongate shaft 610 may incorporate the features and may use the methods described with respect to the modulation system 10 and elongate shaft 14 illustrated in FIGS. 2-4. The system 600 may include an inflatable balloon 614 disposed on or adjacent to the elongate shaft 610. In some embodiments, the balloon 614 may be secured directly to the elongate shaft 610 in any manner desired. In other embodiments, the balloon 614 may be secured to the elongate shaft 610 in such a way that the balloon 614 does not directly contact the elongate shaft 610. It is contemplated that the balloon 614 may be connected and operated as discussed with respect to the balloons 32, 34 illustrated in FIGS. 2-4. The balloon 614 may be sized and shaped to occlude a desired portion of the lumen 622. In some embodiments, the balloon may be sized and shaped such that in the inflated state, the balloon 614 partially covers one or more electrodes 630.

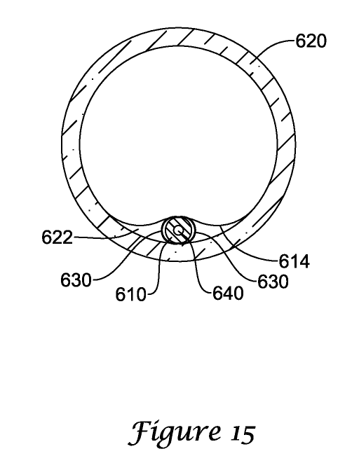

The system 600 may further include one or more electrodes 630 disposed on the outer surface of the elongate shaft 610. In some instances the one or more electrodes 630 may be positioned such that they are not in direct contact with the vessel wall 620 as shown more clearly in FIG. 15. The electrode(s) 630 may be formed and attached to the shaft 610 in the manner described with respect to electrodes 40 shown in FIGS. 2-4. The electrode(s) 630 may be formed of any suitable material, shape, and size such as those described with respect to electrodes 40 shown in FIGS. 2-4. While the electrode(s) 630 are shown as disposed on the elongate shaft 610, it is contemplated that in some embodiments, the electrode(s) 630 may be disposed on the surface of the balloon 614. In other embodiments, a region of the balloon 614 may be made conductive.

FIG. 15 illustrates an end view of the illustrative modulation system 600 shown in FIG. 14 disposed within a vessel 620. In some instances, the elongate shaft 610 may include a lumen 640 for receiving a guidewire or other device. While the system 600 is illustrated as including two electrodes 630, it is contemplated the system may include any number of electrodes 630 desired, for example one, two, three, four, or more. In some instances, the electrodes 630 may be positioned on the distal end region 612 of the elongate shaft 610 approximately 180° from one another. However, the electrodes 630 may be positioned in any radial or circumferential position desired. Further, in some embodiments, electrodes 630 may be placed at different longitudinal positions along the length of the elongate shaft 610. The balloon 614 may be sized and shaped to space the electrodes 630 a distance from the vessel wall 620 in an off-the-wall or non-contact arrangement. In some instances, the diameter of the balloon 614 may be larger than the distance between the elongate shaft 610 and the vessel wall 620 such that when expanded the balloon 614 extends partially around the elongate shaft 610 taking on a kidney bean type shape. The balloon 614 may further maintain consistent spacing between the vessel wall 620 and the electrodes 630 such that fluid flow past the electrodes 630 may be preserved. The single balloon 614 may position the elongate shaft 610 close to the vessel wall 620 such that the electrodes 630 are positioned closer to one side of the vessel 620.

While not explicitly shown, the electrodes 630 may be connected to a control unit (such as control unit 18 in FIG. 1) by electrical conductors. Once the modulation system 600 has been advanced to the treatment region, energy may be supplied to the electrodes 630. The amount of energy delivered to the electrodes 630 may be determined by the desired treatment. For example, more energy may result in a larger, deeper lesion. In some embodiments, it may be desired to achieve the hottest, deepest lesion beyond the vessel wall while minimizing the temperature at the surface of the vessel wall 620. The temperature at the surface of the vessel wall 620 may be a function of the power used as well as the fluid flow through the vessel 620. In some instances, the increased velocity of fluid flow resulting from the partial vessel occlusion may allow more power to be used during treatment. While the current density traveling between, for example, electrode 630 and ground electrode 20 (shown in FIG. 1) may result in the heating of adjacent fluid and tissue, there may be negligible resistance in the electrode 630 such that the electrode 630 does not get hot.

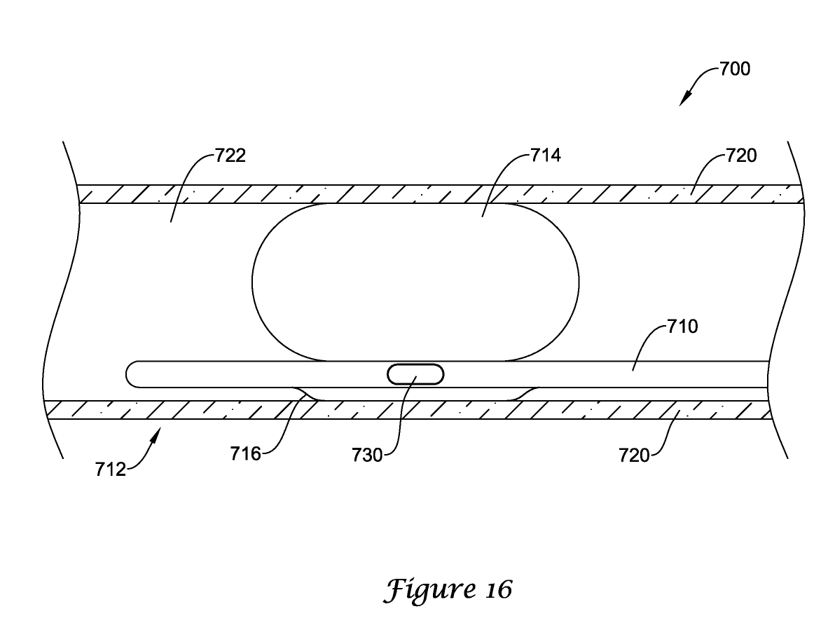

FIG. 16 is another illustrative embodiment of a distal end of a renal nerve modulation system 700 disposed within a body lumen 722 having a vessel wall 720. The system 700 may include an elongate shaft 710 having a distal end region 712. It is contemplated that the system 700 and elongate shaft 710 may incorporate the features and may use the methods described with respect to the modulation system 10 and elongate shaft 14 illustrated in FIGS. 2-4. The system 700 may include an inflatable balloon 714 disposed on or adjacent to the elongate shaft 710. In some embodiments, the balloon 714 may be secured directly to the elongate shaft 710 in any manner desired. In other embodiments, the balloon 714 may be secured to the elongate shaft 710 in such a way that the balloon 714 does not directly contact the elongate shaft 710. It is contemplated that the balloon 714 may be connected and operated as discussed with respect to the balloons 32, 34 illustrated in FIGS. 2-4. The balloon 714 may be sized and shaped to occlude a desired portion of the lumen 722. The system 700 may further include a spacing mechanism 716 attached to the distal end region 712 of the elongate shaft 710. In some instances, the balloon 714 and the spacing mechanism 716 may be positioned on the distal end region 712 of the elongate shaft 710 approximately 180° from one another. However, it is contemplated the balloon 714 and the spacing mechanism 716 may have any radial or circumferential arrangement desired. In some embodiments, the spacing mechanism 716 may be an insulated elastic wire. However, it is contemplated that the spacing mechanism 716 may be formed of any non-electrically conductive material. The spacing mechanism 716 may contact only a portion of the vessel wall 720 such that RF current may pass through that portion of the vessel wall 720.

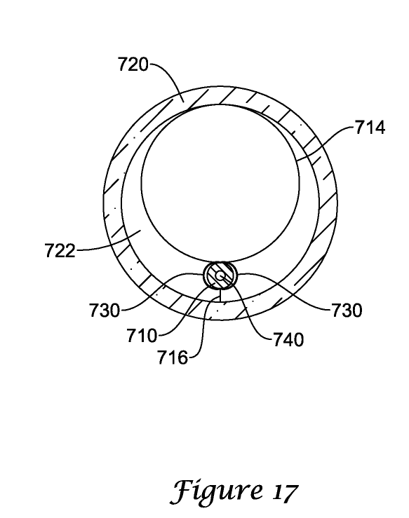

The system 700 may further include one or more electrodes 730 disposed on the outer surface of the elongate shaft 710. In some instances the one or more electrodes 730 may be positioned such that they are not in direct contact with the vessel wall 720 as shown more clearly in FIG. 17. The electrode(s) 730 may be formed and attached to the shaft 710 in the manner described with respect to electrodes 40 shown in FIGS. 2-4. The electrode(s) 730 may be formed of any suitable material, shape, and size such as those described with respect to electrodes 40 shown in FIGS. 2-4. While the electrode(s) 730 are shown as disposed on the elongate shaft 710, it is contemplated that in some embodiments, the electrode(s) 730 may be disposed on the surface of the balloon 714. In other embodiments, a region of the balloon 714 may be made conductive. In some embodiments, the electrodes 730 may be a single electrode disposed around the entire perimeter of the elongate shaft 710. A single electrode 730 may allow for 360° ablation. Thus, the elongate shaft 710 may not require repositioning.

FIG. 17 illustrates an end view of the illustrative modulation system 700 disposed within a vessel 720. In some instances, the elongate shaft 710 may include a lumen 740 for receiving a guidewire or other device. While the system 700 is illustrated as including two electrodes 730, it is contemplated the system may include any number of electrodes 730 desired, for example one, two, three, four, or more. In some instances, the electrodes 730 may be positioned on the distal end region 712 of the elongate shaft 710 approximately 180° from one another. The balloon 714 may be sized and shaped to space the electrodes 730 a distance from the vessel wall 720 in an off-the-wall or non-contact arrangement. The balloon 714 may further maintain consistent spacing between the vessel wall 720 and the electrodes 730 such that fluid flow past the electrodes 730 may be preserved. The single balloon 714 may position the elongate shaft 710 close to the vessel wall 720 such that the electrodes 730 are positioned closer to one side of the vessel 720. The spacing mechanism 716 may allow fluid flow to along the vessel wall 720 during the ablation process allowing for more effective cooling.

While not explicitly shown, the electrodes 730 may be connected to a control unit (such as control unit 18 in FIG. 1) by electrical conductors. Once the modulation system 700 has been advanced to the treatment region, energy may be supplied to the electrodes 730. The amount of energy delivered to the electrodes 730 may be determined by the desired treatment. For example, more energy may result in a larger, deeper lesion. In some embodiments, it may be desired to achieve the hottest, deepest lesion beyond the vessel wall while minimizing the temperature at the surface of the vessel wall 720. The temperature at the surface of the vessel wall 720 may be a function of the power used as well as the fluid flow through the vessel 720. In some instances, the increased velocity of fluid flow resulting from the partial vessel occlusion may allow more power to be used during treatment. While the current density traveling between, for example, electrode 730 and ground electrode 20 (shown in FIG. 1) may result in the heating of adjacent fluid and tissue, there may be negligible resistance in the electrode 730 such that the electrode 730 does not get hot.

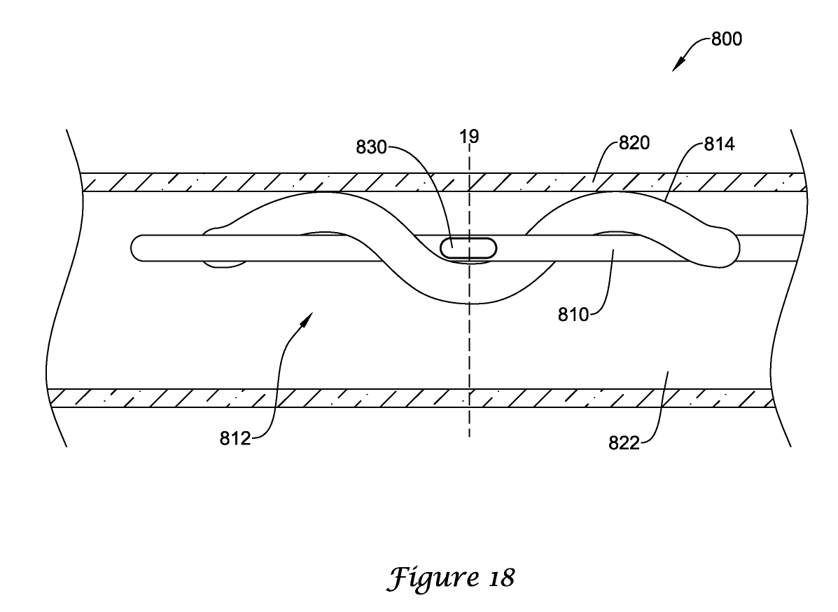

FIG. 18 is another illustrative embodiment of a distal end of a renal nerve modulation system 800 disposed within a body lumen 822 having a vessel wall 820. The system 800 may include an elongate shaft 810 having a distal end region 812. It is contemplated that the system 800 and elongate shaft 810 may incorporate the features and may use the methods described with respect to the modulation system 10 and elongate shaft 14 illustrated in FIGS. 2-4. The system 800 may include an inflatable balloon 814 disposed on or adjacent to the elongate shaft 810. The balloon 814 may have a spiral shape configured to wrap around the perimeter of the elongate shaft 810. The spiral balloon 814 may provide controlled spacing for fluid flow between an electrode 830 and the vessel wall 820. The spiral shape of the balloon 814 may provide a spiral path for fluid flow thus increasing heat transfer away from the treatment region. This may reduce negative side effects of nerve ablation, such as, but not limited to thermal injury to the vessel wall, blood damage, clotting and/or protein fouling of the electrode. In some embodiments, the balloon 814 may be secured directly to the elongate shaft 810 in any manner desired. In other embodiments, the balloon 814 may be secured to the elongate shaft 810 in such a way that the balloon 814 does not directly contact the elongate shaft 810. It is contemplated that the balloon 814 may be connected and operated as discussed with respect to the balloons 32, 34 illustrated in FIGS. 2-4.

The system 800 may further include one or more electrodes 830 disposed on the outer surface of the elongate shaft 810. In some instances the one or more electrodes 830 may be positioned such that they are not in direct contact with the vessel wall 820 as shown more clearly in FIG. 8. The electrode(s) 830 may be formed and attached to the shaft 810 in the manner described with respect to electrodes 40 shown in FIGS. 2-4. The electrode(s) 830 may be formed of any suitable material, shape, and size such as those described with respect to electrodes 40 shown in FIGS. 2-4. While the electrode(s) 830 are shown as disposed on the elongate shaft 810, it is contemplated that in some embodiments, the electrode(s) 830 may be disposed on the surface of the balloon 814. In other embodiments, a region of the balloon 814 may be made conductive. In some embodiments, the electrodes 830 may be a single electrode disposed around the entire perimeter of the elongate shaft 810. A single electrode 830 may allow for 360° ablation. Thus, the elongate shaft 810 may not require repositioning.

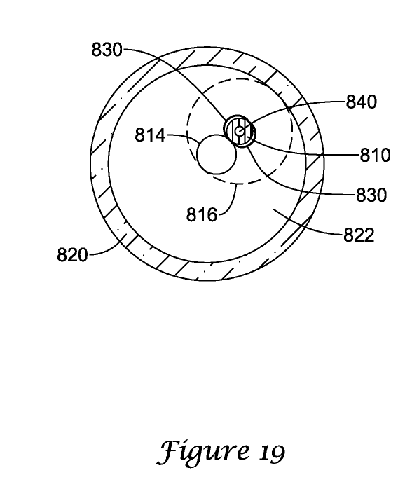

FIG. 19 illustrates cross-section of the illustrative modulation system 800 disposed within a vessel 820 taken at line 19 in FIG. 18. The modulating system 800 may occupy a relatively small portion of the vessel lumen 822 as indicted by the dashed line 816 in FIG. 19. In some instances, the elongate shaft 810 may include a lumen 840 for receiving a guidewire or other device. While the system 800 is illustrated as including two electrodes 830, it is contemplated the system may include any number of electrodes 830 desired, for example one, two, three, four, or more. In some instances, the electrodes 830 may be positioned on the distal end region 812 of the elongate shaft 810 approximately 180° from one another. The balloon 814 may be sized and shaped to space the electrodes 830 a distance from the vessel wall 820 in an off-the-wall or non-contact arrangement. The balloon 814 may further maintain spacing between the vessel wall 820 and the electrodes 830 such that fluid flow past the electrodes 830 may be preserved.

While not explicitly shown, the electrodes 830 may be connected to a control unit (such as control unit 18 in FIG. 1) by electrical conductors. Once the modulation system 800 has been advanced to the treatment region, energy may be supplied to the electrodes 830. The amount of energy delivered to the electrodes 830 may be determined by the desired treatment. For example, more energy may result in a larger, deeper lesion. In some embodiments, it may be desired to achieve the hottest, deepest lesion beyond the vessel wall while minimizing the temperature at the surface of the vessel wall 820. The temperature at the surface of the vessel wall 820 may be a function of the power used as well as the fluid flow through the vessel 820. In some instances, the increased velocity of fluid flow resulting from the partial vessel occlusion may allow more power to be used during treatment. While the current density traveling between, for example, electrode 830 and ground electrode 20 (shown in FIG. 1) may result in the heating of adjacent fluid and tissue, there may be negligible resistance in the electrodes 830 such that the electrodes 830 do not get hot.

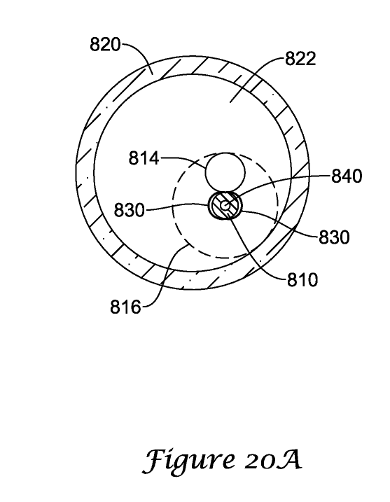

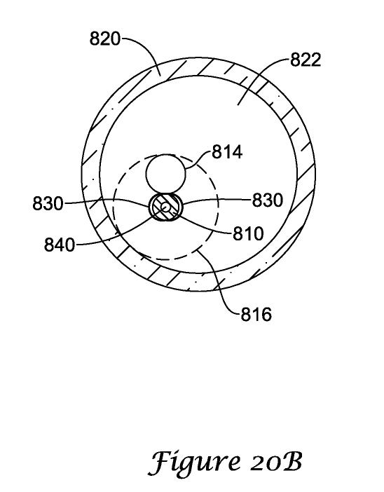

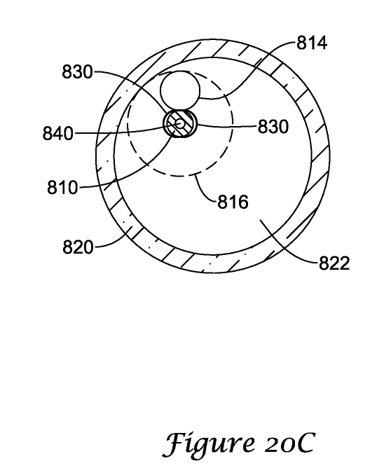

When the balloon 814 is inflated, the modulation system 800 may not extend across the entire lumen 822 of the vessel 820. The elongate shaft 810 may need to be manipulated to provide complete ablation around the perimeter of the vessel 820. The elongate shaft 810 may be directed towards different axial and circumferential locations of the vessel wall 820 by manual torquing, a guide catheter, stylet, active bending mechanism, or other means. As shown in FIGS. 20A-20C, the elongate shaft 810 may be manipulated such that the electrode(s) 830 are placed in close proximity to different portions of the vessel wall 820 during ablation.

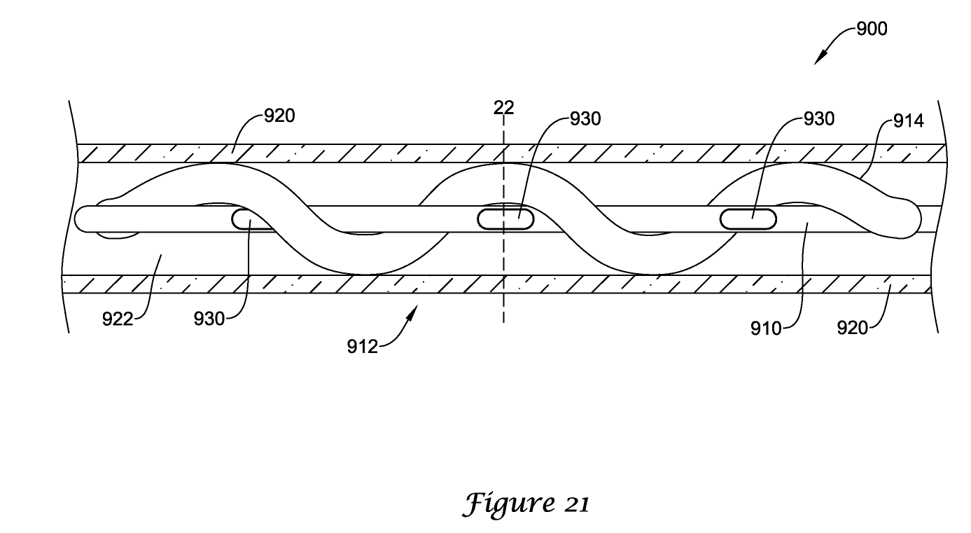

FIG. 21 is another illustrative embodiment of a distal end of a renal nerve modulation system 900 disposed within a body lumen 922 having a vessel wall 920. The system 900 may include an elongate shaft 910 having a distal end region 912. It is contemplated that the system 900 and elongate shaft 910 may incorporate the features and may use the methods described with respect to the modulation system 10 and elongate shaft 14 illustrated in FIGS. 2-4.

The system 900 may include an inflatable balloon 914 disposed on or adjacent to the elongate shaft 910. The balloon 914 may have a spiral shape configured to wrap around the perimeter of the elongate shaft 910. The spiral balloon 914 may provide controlled spacing for fluid flow between an electrode 930 and the vessel wall 920. The spiral shape of the balloon 914 may provide a spiral path for fluid flow thus increasing heat transfer away from the treatment region. This may reduce negative side effects of nerve ablation, such as, but not limited to thermal injury to the vessel wall, blood damage, clotting and/or protein fouling of the electrode. The spiral balloon 914 may partially occlude the vessel lumen 922 thus increasing the velocity of blood flow in a region proximate the desired treatment area. The increased velocity of blood flow may increase the convective cooling of the blood and tissue surrounding the treatment area and reducing artery wall thermal injury, blood damage, and/or clotting. In some embodiments, when inflated the balloon 914 may partially deform the elongate shaft 910 to induce a corresponding spiral in the elongate shaft 910. This may bend the shaft 910 such that the electrodes 930 are moved closer to the vessel wall 920. In some embodiments, the balloon 914 may be secured directly to the elongate shaft 910 in any manner desired. In other embodiments, the balloon 914 may be secured to the elongate shaft 910 in such a way that the balloon 914 does not directly contact the elongate shaft 910. It is contemplated that the balloon 914 may be connected and operated as discussed with respect to the balloons 32, 34 illustrated in FIGS. 2-4.

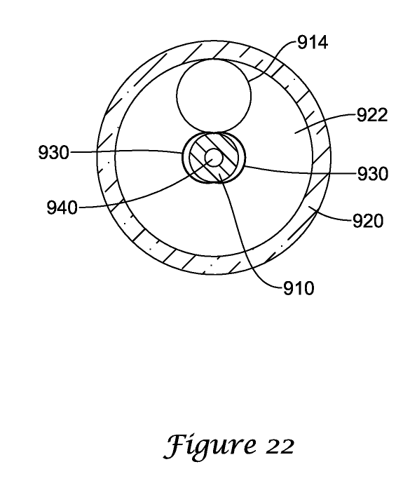

The system 900 may further include one or more electrodes 930 disposed on the outer surface of the elongate shaft 910. The system 900 may include electrodes 930 positioned in different longitudinal locations along the elongate shaft 910. Such an orientation may allow a user to perform ablation on a longer region without repositioning the elongate shaft 910. The electrodes 930 may be energized simultaneously, sequentially, or in a bipolar arrangement as described with respect to electrodes 40 shown in FIGS. 2-4. In some instances, the one or more electrodes 930 may be positioned such that they are not in direct contact with the vessel wall 920 as shown more clearly in FIG. 22. The electrode(s) 930 may be formed and attached to the shaft 910 in the manner described with respect to electrodes 40 shown in FIGS. 2-4. The electrode(s) 930 may be formed of any suitable material, shape, and size such as those described with respect to electrodes 40 shown in FIGS. 2-4. While the electrode(s) 930 are shown as disposed on the elongate shaft 910, it is contemplated that in some embodiments, the electrode(s) 930 may be disposed on the surface of the balloon 914. In other embodiments, a region of the balloon 914 may be made conductive. In some embodiments, the electrodes 930 may be a single electrode disposed around the entire perimeter of the elongate shaft 910. A single electrode 930 may allow for 360° ablation. Thus, the elongate shaft 910 may not require repositioning.

FIG. 22 illustrates a cross-section of the illustrative modulation system 900 disposed within a vessel 920 taken at line 22 in FIG. 21. In some instances, the elongate shaft 910 may include a lumen 940 for receiving a guidewire or other device. While the system 900 is illustrated as including two electrodes 930 at a given longitudinal location, it is contemplated the system may include any number of electrodes 930 desired at each longitudinal location, for example one, two, three, four, or more. In some instances, the electrodes 930 may be positioned on the distal end region 912 of the elongate shaft 910 approximately 180° from one another. The balloon 914 may be sized and shaped to space the electrodes 930 a distance from the vessel wall 920 in an off-the-wall or non-contact arrangement. The balloon 914 may further maintain spacing between the vessel wall 920 and the electrodes 930 such that fluid flow past the electrodes 930 may be preserved.

While not explicitly shown, the electrodes 930 may be connected to a control unit (such as control unit 18 in FIG. 1) by electrical conductors. Once the modulation system 900 has been advanced to the treatment region, energy may be supplied to the electrodes 930. The amount of energy delivered to the electrodes 930 may be determined by the desired treatment. For example, more energy may result in a larger, deeper lesion. In some embodiments, it may be desired to achieve the hottest, deepest lesion beyond the vessel wall while minimizing the temperature at the surface of the vessel wall 920. The temperature at the surface of the vessel wall 920 may be a function of the power used as well as the fluid flow through the vessel 920. In some instances, the increased velocity of fluid flow resulting from the partial vessel occlusion may allow more power to be used during treatment. While the current density traveling between, for example, electrode 930 and ground electrode 20 (shown in FIG. 1) may result in the heating of adjacent fluid and tissue, there may be negligible resistance in the electrodes 930 such that the electrodes 930 do not get hot. In some instances, the balloon 914 may direct current away from the balloon 914 and towards the vessel wall 920 opposite the balloon.

While the methods of use have been described with respect to the various embodiments, a brief summary of an illustrative use will be described using the modulation system 10 of FIGS. 2-4. However, any of the modulations systems 100, 200, 300, 400, 500, 600, 700, 800, or 900 described with respect to FIGS. 5-22 may be operated in the following manner.

The modulation system 10 may be advanced through the vasculature in any manner known in the art. For example, system 10 may include a guidewire lumen 36 to allow the system 10 to be advanced over a previously located guidewire. In some embodiments, the modulation system 10 may be advanced, or partially advanced, within a guide sheath such as the sheath 16 shown in FIG. 1. The first and second balloons 32, 34 may be deflated during introduction, advancement, and removal of the system 10. Once the distal end region 30 of the modulation system 10 has been placed adjacent to the desired treatment area, the balloons 32, 34 may be inflated to partially occlude the vessel lumen 52. Once inflated the balloons 32, 34 may reduce the cross-sectional area of the vessel and may maintain consistent spacing between the vessel wall 50 and the electrode 40. While not explicitly shown, the electrodes 40 may be connected to a control unit (such as control unit 18 in FIG. 1) by electrical conductors. Once the modulation system 10 has been advanced to the treatment region, energy may be supplied to the electrodes 40. The amount of energy delivered to the electrodes 40 may be determined by the desired treatment. Once ablation has been completed for the desired region, the balloons 32, 34 may be deflated and the elongate shaft 14 rotated by 90°. Once the elongate shaft 14 has been repositioned, the balloons 32, 34 may be reinflated and energy may once again be delivered to the electrodes 40. The number of times the elongate shaft 14 is rotated at a given longitudinal location may be determined by the number and size of the electrodes 40 on the elongate shaft 14. For example, an elongate shaft 14 including only a single electrode 40 sized and shaped similar to the one shown in FIGS. 2-4 may need to be rotated multiple times to achieve 360° ablation. However, in some embodiments, the electrodes 40 may be a single electrode disposed around the entire perimeter of the elongate shaft 14. Such an electrode 40 may allow for 360° ablation. Thus, the elongate shaft 14 may not require repositioning. Once a particular location has been ablated, it may be desirable to perform further ablation at different longitudinal locations. The balloons 32, 34 may be deflated at the treatment region to allow for longitudinal displacement of the modulation system 10. Once the elongate shaft 14 has been repositioned, the balloons 32, 34 may be reinflated and energy may once again be delivered to the electrodes 40. Once ablation has been completed for the desired region, the balloons 32, 34 may be deflated and the elongate shaft 14 rotated by 90°. Once the elongate shaft 14 has been repositioned, the balloons 32, 34 may be reinflated and energy may once again be delivered to the electrodes 40. The number of times the elongate shaft 14 is rotated at a given longitudinal location may be determined by the number and size of the electrodes 40 on the elongate shaft 14. This process may be repeated at any number of longitudinal locations desired. It is contemplated that in some embodiments, the system 10 may include electrodes 40 positioned at various positions along the length of the modulation system 10 such that a larger region may be treated without longitudinal displacement of the elongate shaft 14. Once the ablation process is complete, the balloons 32, 34 may be deflated and the modulation system 10 removed from the vasculature.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A system for nerve modulation, comprising
an elongate shaft having a proximal end region and a distal end region;
a first inflatable balloon having a proximal end and a distal end, the first inflatable balloon disposed proximate the distal end region of the elongate shaft;
an outer member having an inner surface and an outer surface, the outer member disposed over the first inflatable balloon and extending from the proximal end to the distal end thereof; and
a nerve modulation element disposed on the outer member.

2. The system of claim 1, further comprising a second inflatable balloon having a proximal end and a distal end, the second balloon disposed adjacent to the first inflatable balloon.

3. The system of claim 2, wherein the second balloon is positioned approximately 180° from the first inflatable balloon on the elongate shaft.

4. The system of claim 1, further comprising two nerve modulation elements.

5. The system of claim 4, wherein one nerve modulation element is disposed on a first side of the outer member and one nerve modulation element is disposed on a second side of the outer member.

6. The system of claim 1, wherein the nerve modulation element is an electrode.

7. The system of claim 1, wherein the outer member includes an open region defining a window.

8. The system of claim 7, wherein the nerve modulation element is attached to the inner surface of the outer member such that at least a portion of the nerve modulation element is visible through the window.

9. The system of claim 1, further comprising a support means attached to the nerve modulation element.

10. The system of claim 1, further comprising a vent in outer member.

11. The system of claim 10, wherein the vent extends between the elongate shaft and the outer member

12. The system of claim 10, wherein the vent comprises microscopic holes in the outer member.

13. The system of claim 12, further comprising microscopic holes in the first balloon.

14. The system of claim 1, wherein the nerve modulation element has an oblong shape.

15. An intravascular nerve ablation system comprising
an elongate shaft having a proximal end and distal end and a lumen extending therebetween;
a first inflatable balloon having a proximal end and a distal end, the first balloon disposed at a first position proximate the distal end of the elongate shaft;
a second inflatable balloon having a proximal end and a distal end, the second balloon disposed at a second position proximate the distal end of the elongate shaft;
a third balloon disposed over the first balloon and the second balloon and extending from the proximal ends of the first and second balloons to the distal ends of first and second balloons; and
a first electrode positioned on a first surface of the third balloon.

16. The system of claim 15, further comprising a second electrode positioned on a second surface of the third balloon.

17. The system of claim 15, wherein the elongate shaft further includes at least one inflation lumen in fluid communication with the first and second inflatable balloons.

18. The system of claim 15, further comprising an opening formed in at least one surface of the third balloon and the first electrode is secured to the third balloon adjacent the opening such that at least a portion of the electrode is visible through the opening.

19. The system of any of claim 15, wherein the electrode is formed directly on the surface of the third balloon.

20. An intravascular nerve ablation system comprising:
an elongate shaft having a proximal end and distal end and a lumen extending therebetween;
a first inflatable balloon having a proximal end and a distal end, the first balloon disposed at a first position proximate the distal end of the elongate shaft;
a second inflatable balloon having a proximal end and a distal end, the second balloon disposed at a second position proximate the distal end of the elongate shaft;
a third balloon having an inner surface and an outer surface disposed over the first balloon and the second balloon and extending from the proximal ends of the first and second balloons to the distal ends of first and second balloons;
a first opening defined in a first side of the third balloon extending from the inner surface to the outer surface;
a second opening defined in a second side of the third balloon extending from the inner surface to the outer surface;
a first electrode attached to the inner surface of the third balloon such that at least a portion of the electrode is visible through the first opening; and
a second electrode attached to the inner surface of the third balloon such that at least a portion of the electrode is visible through the second opening;
wherein the first position is 180° from the second position, the first opening is 180° from the second opening, and the first opening is 90° from the first position.
